(19) United States
(12) Patent Application Publication
(10) Pub. No.: US 2025/0107992 A1
(43) Pub. Date: Apr. 3, 2025

(54) COSMETIC COMPOSITION

(71) Applicant: AJINOMOTO CO., INC., Tokyo (JP)

(72) Inventors: Eri Higashiura, Kanagawa (JP); Kenichi Mori, Kanagawa (JP); Yoshinobu Takino, Kanagawa (JP)

(73) Assignee: AJINOMOTO CO., INC., Tokyo (JP)

(21) Appl. No.: 18/354,140

(22) Filed: Jul. 18, 2023

(51) Int. Cl. A61K 8/68; A61K 31/164; A61Q 19/00

(52) U.S. Cl. CPC A61K 8/68; A61K 31/164; A61Q 19/007; A61K 2800/5922

(57) ABSTRACT

A composition, such as a cosmetic composition, is provided. The composition contains ceramide NH having an acyl chain length of C18.

This application is a Continuation of, and claims priority under 35 U.S.C. § 120 to, International Application No. PCT/JP2022/001984, filed Jan. 20, 2022, and claims priority therethrough under 35 U.S.C. § 119 to Japanese Patent Application No. 2021-006810, filed Jan. 20, 2021, the entireties of which, as well as all citations cited herein, are incorporated by reference herein.

### BACKGROUND

### Technical Field

The present invention relates to a composition, such as a cosmetic composition.

### Background Art

A ceramide is a compound having a structure in which a sphingoid base and a fatty acid are covalently linked to each other via an amide bond. That is, a ceramide includes a sphingoid base moiety, namely, an alkyl chain, and a fatty acid moiety, namely, an acyl chain, which moieties are covalently linked to each other via an amide bond. Various groups of ceramides have been known depending on the types of the sphingoid base and fatty acid (Masukawa Y. et al., J Lipid Res. 2008 July; 49(7):1466-76; “Characterization of overall ceramide species in human stratum corneum”). Some ceramides have been used as ingredients for pharmaceuticals, cosmetics, and so forth.

Ceramide NH is a ceramide of 6-hydroxysphingosine (hereafter, also referred to as “6HS”. That is, ceramide NH is a ceramide of which the sphingoid base moiety is 6HS. That is, ceramide NH has a 6HS moiety, namely, an alkyl chain, and a fatty acid moiety, namely, an acyl chain, which moieties are covalently linked to each other via an amide bond. As ceramide NHs, those having different lengths of acyl chains such as C18 have been known (See Masukawa et al., cited above, and Vielhaber G. et al., J Invest Dermatol. 2001 November; 117(5):1126-36. “Localization of ceramide and glucosylceramide in human epidermis by immunogold electron microscopy”).

However, the effects of the lengths of acyl chains on functions of ceramide NHs have not been previously reported.

### SUMMARY

An aspect of the present invention is to provide a composition, such as a cosmetic composition.

Described herein is the finding that ceramide NH having an acyl chain length of C18 can significantly improve skin condition as compared with ceramide NHs having other acyl chain lengths.

It is an aspect of the present invention to provide a composition for improving the condition of an organism's body surface, the composition comprising a ceramide NH comprising a C18 acyl chain.

It is a further aspect of the present invention to provide the composition as described above, wherein the body surface is skin.

It is a further aspect of the present invention to provide the composition as described above, wherein the composition is able to improve a lamellar structure of skin, barrier function of skin, or moisturizing capacity of skin. It is a further aspect of the present invention to provide the composition as described above, wherein the composition is able to prevent or treat a symptom relating to deterioration in skin condition.

It is a further aspect of the present invention to provide the composition as described above, wherein the symptom is selected from the group consisting of rough skin, dry skin, squama, scale, desquamation, dermatitis, psoriasis, and combinations thereof.

It is a further aspect of the present invention to provide the composition as described above, wherein the composition is formulated as a cosmetic composition or a pharmaceutical composition.

It is a further aspect of the present invention to provide the composition as described above, wherein the composition further contains an ingredient formulated for a cosmetic or pharmaceutical.

It is a further aspect of the present invention to provide the composition as described above, wherein the ingredient formulated for a cosmetic or pharmaceutical is selected from the group consisting of a binder, disintegrant, lubricant, stabilizer, diluent, surfactant, pH adjuster, vitamin, mineral, perfume, pigment, preservative, terpenoid, lipid, fatty acid, alcohol, water, antioxidant, anti-inflammatory, moisturizing ingredient, anti-ageing ingredient, anti-cellulite ingredient, skin whitening ingredient, skin tanning ingredient, UV filter, and combinations thereof.

It is a further aspect of the present invention to provide the composition as described above, wherein said ingredient formulated for a cosmetic or pharmaceutical is selected from the group consisting of a terpenoid, a lipid, a fatty acid, and combinations thereof.

It is a further aspect of the present invention to provide the composition as described above, wherein the terpenoid is selected from the group consisting of cholesterol, cholesterol sulfate, squalene, pristane, and combinations thereof.

It is a further aspect of the present invention to provide the composition as described above, wherein the lipid is selected from the group consisting of triolein, phosphatidylethanolamine, and combinations thereof.

It is a further aspect of the present invention to provide the composition as described above, wherein the fatty acid is selected from the group consisting of lauric acid, myristic acid, palmitic acid, stearic acid, arachidic acid, behenic acid, lignoceric acid, oleic acid, linolenic acid, and combinations thereof.

It is a further aspect of the present invention to provide the composition as described above, wherein the ceramide NH comprises a C18:1 alkyl chain and a C18 acyl chain.

It is a further aspect of the present invention to provide the composition as described above, wherein the ceramide NH comprises a C18:1 alkyl chain and a C18: 0 acyl chain.

It is a further aspect of the present invention to provide the composition as described above, wherein the C18:1 alkyl chain has hydroxyl groups at positions C1, C3, and C6 only.

### BRIEF DESCRIPTION OF DRAWINGS

FIG. 1 is a photograph of Maltese cross patterns of C14-CeramideNH, that is, a ceramide NH having an acyl chain length of C14.

FIG. 2 is a photograph of Maltese cross patterns of C16-CeramideNH, that is, ceramide NH having an acyl chain length of C16.

FIG. 3 is a photograph of Maltese cross patterns of C18-CeramideNH, that is, ceramide NH having an acyl chain length of C18.

FIG. 4 is a photograph showing Maltese cross patterns of C22-CeramideNH, that is, ceramide NH having an acyl chain length of C22.

FIG. 5 is a diagram showing the effects of ceramide NHs having different acyl chain lengths on skin condition recovery. C14, C16, C18, and C22 represent acyl chain lengths of ceramide NHs.

### DETAILED DESCRIPTION OF THE EXEMPLARY EMBODIMENTS

The various explanations of the exemplary embodiments as described herein may be applied independently or in any appropriate combination, unless otherwise stated.

### <1>Composition

The composition as described herein includes a ceramide NH having an acyl chain length of C18. This ceramide NH is referred to as “active ingredient”.

The composition may be, for example, a cosmetic composition or a pharmaceutical composition. The composition may particularly be a cosmetic composition. The term a “cosmetic composition” refers to a composition which is used for cosmetic use, and it may be synonymous with a cosmetic. The term a “pharmaceutical composition” refers to a composition which is used for pharmaceutical use, and it may be synonymous with a pharmaceutical. Both the terms “cosmetic composition” and “pharmaceutical composition” may include quasi drugs such as medicinal cosmetics, unless otherwise stated.

The composition can be used by, for example, applying the same to an organism. The phrase “application of the composition to an organism” may mean that the composition is brought into contact with a desired target or part/portion of an organism so that a desired effect is obtained. The composition can be applied to an organism according to the method as described herein. The application target and application portion of the composition can be appropriately selected according to various conditions such as purpose of the composition. Examples of the organism, namely, the application target of the composition, include mammals. Examples of the mammals include primates such as human, monkey, and chimpanzee; rodents such as mouse, rat, hamster, and guinea pig; and other various mammals such as rabbit, horse, cow, sheep, goat, pig, dog, and cat. Particular examples of mammals include human. Examples of the application target of the composition include a body surface of an organism. Examples of the body surface include skin and body hair. Particular examples of the body surface include skin. The skin may be any part or portion of skin. Examples of the skin include the skin of the face, head, and body. Examples of body include hands, arms, foots, legs, shoulders, breasts, waist, hip, back, and any other parts of body. The body hair may be any part or portion of body hair. Examples of the body hair include head hair.

The organism may be a healthy individual or may be an unhealthy individual. The organism may or may not be, for example, an individual suffering from deterioration in the condition of a body surface, such as skin and body hair. The organism may or may not be, specifically, for example, an individual having a symptom relating to deterioration in the condition of a body surface, such as skin and body hair.

The application target of the composition may be a target in a healthy state or may be a target in an unhealthy state. The application target of the composition may or may not be, for example, a body surface, such as skin and body hair, that is suffering from deterioration in it's condition. The application target of the composition may or may not be, specifically, for example, a body surface, such as skin and body hair, wherein the subject having the skin and body hair suffers from a symptom relating to deterioration in the condition of the skin and body.

Examples of the “desired effect” of the composition include an effect obtainable by, for example, application to an organism, of the active ingredient. That is, the effect obtainable by, for example, application to an organism, of the active ingredient may be obtained by, for example, application to an organism, of the composition. In other words, the composition may have a function of providing the effect obtainable by use, for example, application to an organism, of the active ingredient. The aforementioned descriptions concerning, for example, application to an organism, of the composition can be applied similarly to, for example, application to an organism, of the active ingredient.

By applying the active ingredient to an organism, the condition of the target of the active ingredient may be improved in the organism, namely, an effect of improving the condition of the application target of the active ingredient may be obtained in the organism. Hence, by using the composition, specifically, by applying the composition to an organism, condition of the application target of the active ingredient may be improved in the organism, namely, an effect of improving the condition of the application target of the active ingredient may be obtained in the organism. In other words, the composition has a function of improving the condition of the application target of the active ingredient. Hence, the composition may improve the condition of the application target thereof, such as a cosmetic or pharmaceutical composition for improving the condition of the application target.

Examples of the improvement in the condition include improvement in the condition of a body surface, such as skin and body hair. Particular examples of the improvement in the condition include improvement in skin condition. Hence, the composition may improve the condition of a body surface, such as skin and body hair, for example, a cosmetic or pharmaceutical composition for improving condition of a body surface, such as skin and body hair. The composition may be, particularly, a composition for improving skin condition, for example, a cosmetic or pharmaceutical composition for improving skin condition.

Examples of improvement in skin condition include improvement in a lamellar structure of skin, such as an increase in orderliness of lamellar structure; improvement, such as increase, in a barrier function of skin; and improvement, such as increase, in a moisturizing capacity of skin. Hence, the composition may improve the lamellar structure of skin, the barrier function of skin, or the moisturizing capacity of skin, such as a cosmetic or pharmaceutical composition for improving the lamellar structure of skin, the barrier function of skin, or the moisturizing capacity of skin. Only one of these indicators may be improved, or two or more of these indicators may be improved. Improvement in a certain indicator may improve another indicator. For example, improvement in the lamellar structure may improve a function of skin, such as the barrier function of skin and the moisturizing capacity of skin. In other words, a composition for improving the barrier function of skin or improving the moisturizing capacity of skin may be an embodiment of a composition for improving the lamellar structure of skin. Also, for example, improvement in the barrier function of skin may improve the moisturizing capacity of skin. In other words, a composition for improving the moisturizing capacity of skin may be an embodiment of a composition for improving the barrier function of skin. Specific examples of skin condition include condition of stratum corneum of skin. That is, improvement in the lamellar structure of skin may specifically be improvement in the lamellar structure of stratum corneum of skin. Improvement in the barrier function of skin may specifically be improvement in the barrier function of stratum corneum of skin. Improvement in the moisturizing capacity of skin may specifically be improvement in the moisturizing capacity of stratum corneum of skin.

Methods for measuring improvement in condition of a body surface, such as skin and body hair, are not particularly limited. Improvement in condition of a body surface can be measured by using a parameter reflecting the condition of the body surface as an indicator. For example, examples of parameters reflecting skin condition include transepidermal water loss (TEWL) and order parameter S. That is, improvement in skin condition can be measured as, for example, a decrease in TEWL. TEWL can be measured in a conventional manner. A decrease in TEWL may mean, specifically, for example, improvement in the barrier function of skin. A decrease in TEWL may also mean, specifically, for example, improvement in the moisturizing capacity of skin. Improvement in skin condition can also be measured as, for example, an increase in the order parameter S. The order parameter S can be measured in a conventional manner. The order parameter S can be measured by, for example, the Electron Spin Resonance (ESR) method. The order parameter S can be measured, specifically, for example, as described in the Examples section. The order parameter S represents orderliness of array of fatty acid chains, such as orderliness of lamellar structure, and a higher value of the order parameter S indicates that fatty acid chains are arrayed in a more orderly manner. An increase in the order parameter S may mean, specifically, for example, improvement in the lamellar structure of skin.

It is sufficient that such indicators as exemplified above each are improved when using the active ingredient, specifically, when using the composition as described herein, as compared with when not using the active ingredient, specifically, when not using the composition. The term “improvement” used for each of such indicators as exemplified above, and including when a deterioration in this indicator occurs when not using the active ingredient, shall include a reduction of such deterioration when using the active ingredient, specifically, when using the composition.

By using the active ingredient, specifically, by applying the active ingredient to an organism, improvement in the condition of the application target of the active ingredient may be obtained in the organism. It is expected that improvement in the condition of the application target of the active ingredient enables preventing and/or treating a symptom relating to deterioration in the condition of this target. That is, examples of the effect based on improvement in condition of the application target of the active ingredient include an effect of preventing and/or treating a symptom relating to deterioration in condition of this target. The symptom relating to deterioration in condition of the application target of the active ingredient may or may not be a disease. Examples of the symptom relating to deterioration in condition of the application target of the active ingredient include a symptom caused by deterioration in condition of this portion and a symptom accompanied by deterioration in condition of this portion. Hence, the composition may prevent and/or treat a symptom relating to deterioration in condition of the application target, such as a cosmetic or pharmaceutical composition for preventing and/or treating a symptom relating to deterioration in condition of the application target. The composition for preventing and/or treating a symptom relating to deterioration in condition of the application target thereof may be an embodiment of the composition for improving condition of the application target thereof.

Examples of the deterioration in condition include deterioration in condition of a body surface, such as skin and body hair. Particular examples of the deterioration in condition include deterioration in skin condition. Hence, the composition may be a composition for preventing and/or treating a symptom relating to deterioration in condition of a body surface, such as skin and body hair, for example, a cosmetic or pharmaceutical composition for preventing and/or treating a symptom relating to deterioration in condition of a body surface, such as skin and body hair. The composition may be, particularly, a composition for preventing and/or treating a symptom relating to deterioration in skin condition, such as a cosmetic or pharmaceutical composition for preventing and/or treating a symptom relating to deterioration in skin condition.

Examples of the deterioration in skin condition include deterioration in the lamellar structure of skin, such as decrease in orderliness of lamellar structure; deterioration, such as decrease, in the barrier function of skin; and deterioration, such as decrease, in the moisturizing capacity of skin. Examples of the symptom relating to deterioration in skin condition include rough skin, dry skin, squama (scale), desquamation, dermatitis, and psoriasis.

The active ingredient, specifically, the composition, may be used for a therapeutic purpose, or may be used for a non-therapeutic purpose. That is, such effects as exemplified above each may be obtained for therapeutic purpose or non-therapeutic purpose, unless otherwise stated. In the case of non-therapeutic purpose, the phrase “treatment of a symptom” may be interpreted as “improvement of a symptom”. The term “therapeutic purpose” may mean, for example, that an act on a human body for treatment is included, and particularly, may mean that an act is carried out as a medical act. The term “non-therapeutic purpose” may mean, for example, that an act on a human body for treatment is not included, and particularly, may mean that an act is carried out as a non-medical act. Examples of the non-therapeutic purpose include such purposes as those for health promotion and beauty.

Ceramide NH is a ceramide of 6-hydroxysphingosine (6HS). Ceramide NH may also be referred to as, for example, “ceramide 8”. Each variation of ceramide NH is also referred to as “ceramide NH species”. Each variation of 6HS is also referred to as “6HS species”.

The term “6HS” refers to a long-chain amino alcohol referred to as a sphingoid base, which has such a structure as described below. 6HS includes an alkyl chain, wherein the alkyl chain has an amino group at position C2. That is, the carbon present at either one terminus of the alkyl chain and linked to the aminated carbon (position C2) is regarded as position C1 of the alkyl chain. The alkyl chain has a hydroxyl group at C6 and further has one or more hydroxyl groups. The alkyl chain may have hydroxyl groups, for example, at positions C1, C3, and C6. The alkyl chain may or may not have additional hydroxyl group(s) other than the hydroxyl groups at positions C1, C3, and C6. The alkyl chain may typically have no additional hydroxyl group other than the hydroxyl groups at C1, C3, and C6. The length and the degree of unsaturation of the alkyl chain may vary. The length of alkyl chain (alkyl chain length) may be, for example, C14 to C26, such as C14, C16, C18, C20, C22, C24, and C26. The alkyl chain length may be, for example, particularly, C16, C18, or C20. The alkyl chain length may be, for example, more particularly, C18. The alkyl chain length may be interpreted as the carbon number, that is, the number of carbon atoms, of the alkyl chain. The alkyl chain has an unsaturated double bond at position C4 (namely, between C4 and C5). The unsaturated double bond at position C4 may be, for example, a trans double bond. The alkyl chain may or may not have an additional unsaturated double bond at position other than C4. The alkyl chain may typically have no additional unsaturated double bond at position other than C4. That is, the term “alkyl chain” used for 6HS and ceramide NH refers to one having at least one unsaturated double bond, such as alkenyl and alkadienyl chains, unless otherwise stated. The configurations of chiral centers may or may not be identical to those in the 6HS moiety of a natural ceramide NH. The position C2 may be, for example, 2S. The position C3 may be, for example, 3R. The position C4 may be, for example, 4E (namely trans double bond). The position C6 may be, for example, 6S or 6R. The position C6 may be, particularly, for example, 6R. The configurations of chiral centers may be, for example, 2S, 3R, 4E, and 6R or 2S, 3R, 4E, and 6S. The configurations of chiral centers may be, particularly, for example, 2S, 3R, 4E, and 6R. The number of carbons in the alkyl chain can be indicated as “n”. 6HS having an alkyl chain whose number of carbons is “n” is also referred to as “Cn 6HS” or “Cn-alkyl 6HS”. For example, the term “C18 6HS” collectively refers to 6HS species having an alkyl chain having a length of C18. The number of unsaturated double bonds of the alkyl chain can be indicated as “m”. 6HS having an alkyl chain whose number of carbons is “n” and whose number of unsaturated double bonds is “m” is also referred to as “Cn: m 6HS” or “Cn: m-alkyl 6HS”. Examples of 6HS can include such variant species of 6HS, wherein the variant species have different lengths and/or different degrees of unsaturation. Specific examples of 6HS include C18: 1 6HS, which has a C18 alkyl chain having one unsaturated double bond. More specific examples of 6HS include C18: 1 6HS that has hydroxyl groups at positions C1, C3, and C6 but has no additional hydroxyl group at position other than C1, C3, and C6. Thus, the term “6HS” may collectively refer to such variant species of 6HS, such as the 6HS species exemplified above.

The term “ceramide NH” refers to a compound having a structure in which 6HS and a fatty acid are covalently linked to each other via an amide bond. That is, ceramide NH includes a 6HS moiety (namely, a moiety corresponding to 6HS) and a fatty acid moiety (namely, a moiety corresponding to a fatty acid), wherein the moieties are covalently linked to each other via an amide bond. The 6HS moiety can also be referred to as an “alkyl chain”. The fatty acid moiety can also be referred to as an “acyl chain”. The amide bond may form between the amino group at position C2 of 6HS and a carboxyl group of the fatty acid. The aforementioned descriptions concerning 6HS are similarly applicable to the 6HS moiety. That is, for example, the length and the degree of unsaturation of the alkyl chain, that is, the 6HS moiety, may vary as with those of 6HS. That is, examples of ceramide NH can include ceramides of the 6HS species exemplified above. Specific examples of ceramide NH include ceramides of C18 6HS. More specific examples of ceramide NH include ceramides of C18:1 6HS. Still more specific examples of ceramide NH include ceramides of C18:1 6HS, wherein C18:1 6HS has hydroxyl groups at positions C1, C3, and C6 but has no additional hydroxyl group at position other than C1, C3, and C6. The length and the degree of unsaturation of the acyl chain, that is, the fatty acid moiety, may also vary. The acyl chain length may be, for example, C14 to C26, such as C14, C16, C18, C20, C22, C24, and C26. The acyl chain length may be, for example, particularly, C14, C16, C18, or C22. However, the acyl chain length of the ceramide NH as the active ingredient is C18. The acyl chain length may be interpreted as the carbon number, that is, the number of carbon atoms, of the acyl chain. The acyl chain may be saturated or may be unsaturated. The acyl chain may have one or more unsaturated double bonds. The acyl chain may or may not have a functional group (namely, substituent group). The acyl chain may have one or more functional groups (substituent groups). Examples of the functional group (substituent group) include hydroxy group. However, the acyl chain has no hydroxy group at C2. The carbon constituting the amide bond (formerly, the carbon of the carboxyl group) is regarded as position Cl of the acyl chain. The acyl chain may typically have no hydroxy group. The acyl chain may typically have no functional group (substituent group). Ceramide NH having an alkyl chain whose number of carbons is “n”, that is, ceramide NH comprising a Cn 6HS moiety, is also referred to as “Cn-alkyl ceramide NH”. The number of carbons of the acyl chain can be indicated as “x”. Ceramide NH having an acyl chain whose number of carbons is “x”, that is, ceramide NH comprising a Cx acyl moiety, is also referred to as “Cx-acyl ceramide NH”. The number of unsaturated double bonds of the acyl chain can be indicated as “y”. Ceramide NH having an acyl chain whose number of carbons is “x” and whose number of unsaturated double bonds is “y”, that is, ceramide NH comprising a Cx: y acyl moiety, is also referred to as “Cx:y-acyl ceramide NH”. Ceramide NH having an alkyl chain whose number of carbons is “n” and an acyl chain whose number of carbons is “x”, that is, ceramide NH comprising a Cn 6HS moiety and a Cx acyl moiety, is also referred to as “Cn-alkyl/Cx-acyl ceramide NH”. For example, the term “C18-alkyl ceramide NH” collectively refers to ceramide NHs having an alkyl chain with a length of C18 and having any acyl chain. For example, the term “C18-acyl ceramide NH” collectively refers to ceramide NHs having an acyl chain with a length of C18 and having any alkyl chain. For example, the term “C18-alkyl/C18-acyl ceramide NH” collectively refers to ceramide NHs having an alkyl chain with a length of C18 and an acyl chain with a length of C18. In the case of ceramide NH having an alkyl chain whose number of carbons is “n” and whose number of unsaturated double bonds is “m”, that is, ceramide NH comprising a Cn:m 6HS moiety, the term “Cn” used for the aforementioned name can be rewritten to “Cn:m”. The same shall apply to “Cx” of the acyl chain. For example, the term “C18:1-alkyl/C18: 0-acyl ceramide NH” collectively refers to ceramide NHs having an alkyl chain with a length of C18 having one double bond and a saturated acyl chain with a length of C18. Ceramide NH defined with the aforementioned name may be a single kind of ceramide NH species or may be a combination of two or more kinds of ceramide NH species, unless otherwise stated. For example, Cx-acyl ceramide NH may be a single kind of Cx-acyl ceramide NH species or may be a combination of two or more kinds of Cx-acyl ceramide NH species. Such a combination may be two or more kinds of Cx-acyl ceramide NH species having different alkyl chains and/or different acyl chains, such as alkyl chains having different lengths and/or different degrees of unsaturation and/or acyl chains having different unsaturation degrees. Thus, the term “ceramide NH” may collectively refer to such variant species of ceramide NH, such as the ceramide NH species exemplified above.

As an example of ceramide NH, a typical structure of C18:1-alkyl ceramide NH is shown in the structural formula (I) shown below. The “n” in the formula corresponds to “x-2” in the aforementioned explanations of ceramide NH. That is, for example, this formula when “n” therein is 16 represents a typical structure of C18:1-alkyl/C18:0-acyl ceramide NH.

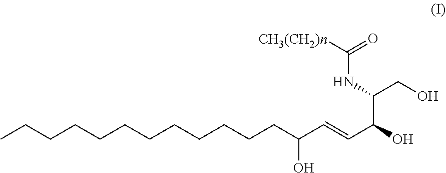

As the active ingredient, ceramide NH having an acyl chain length of C18 (C18-acyl ceramide NH) is selected. The term “C18-acyl ceramide NH” is as described above. The active ingredient (C18-acyl ceramide NH) may be a single kind of C18-acyl ceramide NH species or may be a combination of two or more kinds of C18-acyl ceramide NH species. When the active ingredient is a combination of two or more kinds of C18-acyl ceramide NH species, the term “amount of the active ingredient” refers to the total amount of the combination.

The active ingredient may include, for example, the C18-acyl ceramide NH exemplified above. The active ingredient may include, specifically, for example, C18-alkyl/C18-acyl ceramide NH. The active ingredient may include, more specifically, for example, C18: 1-alkyl/C18-acyl ceramide NH. The active ingredient may include, still more specifically, for example, C18:1-alkyl/C18:0-acyl ceramide NH. The active ingredient may include, still more specifically, for example, C18:1-alkyl/C18-acyl ceramide NH, such as C18:1-alkyl/C18:0-acyl ceramide NH, wherein the alkyl chain has hydroxyl groups at positions C1, C3, and C6 but has no additional hydroxyl group at position other than C1, C3, and C6. The active ingredient may include, still more specifically, for example, C18: 1-alkyl/C18-acyl ceramide NH, such as C18:1-alkyl/C18:0-acyl ceramide NH, wherein the acyl chain has no substituent group. The active ingredient may include, particularly specifically, for example, C18:1-alkyl/C18-acyl ceramide NH, such as C18:1-alkyl/C18:0-acyl ceramide NH, wherein the alkyl chain has hydroxyl groups at positions C1, C3, and C6 but has no additional hydroxyl group at position other than C1, C3, and C6, and the acyl chain has no substituent group. The phrase “the active ingredient includes a certain C18-acyl ceramide NH” means that at least the certain C18-acyl ceramide NH is used as the active ingredient and include when the active ingredient consists of the certain C18-acyl ceramide NH and when the active ingredient consists of a combination of the certain C18-acyl ceramide NH and one or more kinds of other C18-acyl ceramide NH species. For example, phrase “the active ingredient includes a certain C18:1-alkyl/C18-acyl ceramide NH” means that at least C18:1-alkyl/C18-acyl ceramide NH is used as the active ingredient and include when the active ingredient is C18:1-alkyl/C18-acyl ceramide NH and when the active ingredient is a combination of C18:1-alkyl/C18-acyl and one or more kinds of other C18-acyl ceramide NH species. When “the active ingredient includes a certain C18-acyl ceramide NH”, the ratio of the amount of the certain C18-acyl ceramide NH to the total amount of the active ingredient may be, for example, 30% by weight or more, 40% by weight or more, 50% by weight or more, 60% by weight or more, 70% by weight or more, 80% by weight or more, 90% by weight or more, 95% by weight or more, 97% by weight or more, 99% by weight or more, 99.5% by weight or more, 99.9% by weight or more, or 100% by weight.

As the active ingredient, a commercially available C18-acyl ceramide NH may be used, or C18-acyl ceramide NH appropriately prepared and obtained may be used. Methods for producing C18-acyl ceramide NH are not particularly limited. C18-acyl ceramide NH can be produced by, for example, known methods. C18-acyl ceramide NH can be produced by, for example, a chemical synthesis method, enzymatic method, bioconversion method, fermentation method, extraction method, or a combination of these. C18-acyl ceramide NH can be produced by, specifically, for example, conversion from 6HS.

Methods for producing 6HS are not particularly limited. 6HS can be produced by, for example, known methods. 6HS can be produced by, for example, a chemical synthesis method, enzymatic method, bioconversion method, fermentation method, extraction method, or a combination of these. 6HS can be produced by, specifically, for example, the method disclosed in FR2843589A1.

Methods for converting 6HS to C18-acyl ceramide NH are not particularly limited. 6HS can be converted to C18-acyl ceramide NH by, for example, a chemical reaction with a fatty acid (U.S. Pat. No. 5,869,711). The chemical reaction can be carried out under, for example, typical conditions for condensing an amine and a carboxylic acid to form an amide bond. The chemical reaction can be carried out using, specifically, for example, a condensing agent. Examples of the condensing agent include carbodiimides such as water soluble carbodiimides (WSCs). Specific examples of the WSCs include 1-Ethyl-3-(3-dimethylaminopropyl) carbodiimide hydrochloride. The fatty acid is not particularly limited, so long as it provides the acyl chain of C18-acyl ceramide NH to be produced. That is, specific examples of the fatty acid include stearic acid (18:0), oleic acid (18:1), linoleic acid (18:2), and linolenic acid (18:3). Particular examples of the fatty acid include stearic acid (18:0). As 6HS, a single kind of 6HS species may be used, or two or more kinds of 6HS species may be used in combination. As the fatty acid, a single kind of fatty acid may be used, or two or more kinds of fatty acids may be used in combination. When using two or more kinds of 6HS species and/or two or more kinds of fatty acids, a mixture of two or more kinds of C18-acyl ceramide NH species is produced.

As the active ingredient, C18-acyl ceramide NH purified to a desired extent may be used, or a material containing C18-acyl ceramide NH may be used.

The composition may include the active ingredient only or may contain one or more ingredients in addition to the active ingredient. That is, the active ingredient may be used as the only ingredient in the composition or may be used in combination with other ingredients in the composition. Only one additional ingredient the active ingredient may be used, or two or more kinds of ingredients may be used in combination.

The ingredient other than the active ingredient is not particularly limited, so long as an intended effect is obtained. As the ingredient other than the active ingredient, ingredients acceptable depending on the purpose of the composition can be used. Examples of the ingredient other than the active ingredient include physiologically acceptable ingredients. Examples of the physiologically acceptable ingredients include ingredients for a cosmetic or pharmaceutical, such as ingredients generally used for cosmetic use or pharmaceutical use. The term “ingredient for a cosmetic” refers to an ingredient usable as an ingredient in a cosmetic. The term “ingredient for a pharmaceutical” refers to an ingredient usable as an ingredient in a pharmaceutical. Examples of the ingredient other than the active ingredient include binders, disintegrants, lubricants, stabilizers, diluents, surfactants, pH adjusters, vitamins, minerals, perfumes, pigments, preservatives, terpenoids, lipids, fatty acids, alcohols, and water. Particular examples of the ingredient other than the active ingredient include terpenoids, lipids, and fatty acids. Examples of the terpenoids include sterols, squalene, and pristane. Examples of the sterols include cholesterol and cholesterol sulfate. Examples of the lipids include triacylglycerols and glycerophospholipids. Examples of the triacylglycerols include triolein. Examples of the glycerophospholipids include phosphatidylethanolamine. Examples of the fatty acids include lauric acid (12:0), myristic acid (14:0), palmitic acid (16:0), stearic acid (18:0), arachidic acid (20:0), behenic acid (22:0), lignoceric acid (24:0), cerotic acid (26:0), myristoleic acid (14:1), palmitoleic acid (16:1), oleic acid (18:1), linoleic acid (18:2), and linolenic acid (18:3). Particular examples of the fatty acids include lauric acid, myristic acid, palmitic acid, stearic acid, arachidic acid, behenic acid, lignoceric acid, oleic acid, and linolenic acid. More particular examples of the fatty acids include lauric acid, myristic acid, palmitic acid, stearic acid, oleic acid, and linolenic acid. More particular examples of the fatty acids include lauric acid. Specific examples of the ingredient other than the active ingredient also include antioxidants, anti-inflammatoireisturizing ingredients, anti-ageing ingredients, anti-cellulite ingredients, skin whitening ingredients, skin tanning ingredients, and UV filters. Specific examples of the ingredient other than the active ingredient also include ceramides such as ceramide NHs not selected as the active ingredient. As the ingredient other than the active ingredient, for example, one or more ingredients exemplified above may be at least selected. In other words, the ingredient other than the active ingredient may include one or more ingredients exemplified above. The phrase “a certain ingredient is selected as the ingredient other than the active ingredient” or “the ingredient other than the active ingredient includes a certain ingredient” means that at least the certain ingredient is used as the ingredient other than the active ingredient and includes when the ingredient other than the active ingredient is the certain ingredient and when the ingredient other than the active ingredient is a combination of the certain ingredient and one or more kinds of other ingredients. All such ingredients as exemplified above can be used as, for example, ingredients for both a cosmetic and a pharmaceutical.

When the ingredient other than the active ingredient can form a salt, the ingredient other than the active ingredient may be a free compound, a salt thereof, or a combination thereof. For example, examples of salts for acidic groups such as carboxyl group (for example, salts of fatty acids) include ammonium salt; salts with alkaline metal such as sodium and potassium; salts with alkaline earth metal such as calcium and magnesium; and salts with other metals such as aluminum and zinc.

The form of the composition is not particularly limited, so long as an intended effect is obtained. The form of the composition may be set according to various conditions such as the type of the active ingredient, the type(s) of the other ingredient(s), the amount(s) of the ingredient(s), the purpose of the composition, and the mode of administration/application of the composition. The composition may be formulated to an intended form. The composition may be provided, for example, in the form of any cosmetic or pharmaceutical composition applicable to skin. Examples of the cosmetic or pharmaceutical composition, especially, examples of the cosmetic composition, include skin care cosmetics, makeup cosmetics, point makeup cosmetics, hair care products, hair setting agents, hair coloring agents, decolorizing agents, permanent agents, wave agents, body care cosmetics, UV care cosmetics. Examples of the skin care cosmetics include cleansing cosmetics (such as face washes and makeup removers), lotions, serums, packs, massage creams, milky lotions, creams, and essences. Examples of the makeup cosmetics include foundations, concealers, face powders, and make-up bases. Examples of the point makeup cosmetics include lipsticks, blushers, and eye makeups (such as eyeshadows, eyeliners, mascaras, and eyebrows). Examples of the hair care products include shampoos and conditioners (also referred to as rinse or treatment). A hair care product may be a product for, for example, obtaining such an effect as exemplified above in head hair and/or head skin. Examples of the hair setting agents include hair creams, hair waxes, hair liquids, and hair sprays. Examples of the hair coloring agents or decolorizing agents include hair manicures and hair colors. Examples of the body care cosmetics include body creams, soaps, body soaps, hand soaps, and antiperspirants. Examples of the UV care cosmetics include sunscreen cosmetics, suntan cosmetics, self-tanning cosmetics, and after-sun cosmetics. Examples of the cosmetic or pharmaceutical composition, especially, examples of the pharmaceutical composition, include external preparations, such as external skin preparations. Examples of the external preparations include solid agents (such as powders), liquid agents (such as lotions and liniments), sprays, ointments, creams, gels, and patches (such as tapes and poultices). The composition may be provided in a form applicable to skin as it is or may be prepared in a form applicable to skin before use.

The amount(s) of ingredient(s) (namely, the active ingredient and optionally other ingredient(s)) in the composition is/are not particularly limited, so long as the intended effect is obtained. The contained amount(s) of ingredient(s) in the composition can be appropriately set according to various conditions such as the type of the active ingredient, the type(s) of the other ingredient(s), the form of the composition, the purpose of the composition, and the mode of application/administration of the composition.

The amount of the active ingredient in the composition, for example, may be 0.01% by weight or more, 0.1% by weight or more, 1% by weight or more, 5% by weight or more, 10% by weight or more, 15% by weight or more, 20% by weight or more, or 30% by weight or more, may be 100% by weight or less, 99.9% by weight or less, 70% by weight or less, 50% by weight or less, 30% by weight or less, 20% by weight or less, 15% by weight or less, 10% by weight or less, 5% by weight or less, or 1% by weight or less, or may be within a range defined as a non-contradictory combination thereof. The total amount of the active ingredient in the composition may specifically be, for example, 0.01 to 1% by weight, 1 to 5% by weight, 5 to 10% by weight, 10 to 20% by weight, 20 to 30% by weight, or 30 to 50% by weight. The total amount of the active ingredient in the composition may specifically be, for example, 0.01 to 50% by weight, 0.01 to 30% by weight, 0.01 to 10% by weight, or 0.01 to 5% by weight. The total amount of the active ingredient in the composition may specifically be, for example, 1 to 50% by weight, 1 to 30% by weight, 1 to 10% by weight, or 1 to 5% by weight. The total amount of the active ingredient in the composition may specifically be, for example, 5 to 50% by weight, 5 to 30% by weight, or 5 to 10% by weight.

When the composition contains ceramide(s) other than the active ingredient, the ratio of the amount of the active ingredient to the total amount of ceramides in the composition may be, for example, 30% by weight or more, 40% by weight or more, 50% by weight or more, 60% by weight or more, 70% by weight or more, 80% by weight or more, 90% by weight or more, 95% by weight or more, 97% by weight or more, 99% by weight or more, 99.5% by weight or more, or 99.9% by weight or more.

Furthermore, the amount of the active ingredient in the composition may be, for example, such an amount that the application amount of the active ingredient is within a desired range when applying the composition to a body surface, such as skin or body hair.

Incidentally, the amount of active ingredient when using a material containing the active ingredient should be calculated based on the amount of the active ingredient itself in the material.

### <2>Method

The method as described herein is a method that includes a step of applying the active ingredient, namely, ceramide NH having an acyl chain length of C18, to an organism.

By the method, specifically, by applying the active ingredient to an organism, the condition of the application target of the active ingredient can be improved in the organism, that is, an effect of improving the condition of the application target of the active ingredient can be obtained in the organism. Hence, the method may be a method for improving the condition of the application target of the active ingredient.

Improvement in the condition is as described above. That is, the method may be a method for improving condition of a body surface, such as skin and body hair. The method may be, particularly, a method for improving skin condition. The method may be a method for improving the lamellar structure of skin, the barrier function of skin, or the moisturizing capacity of skin. A method for improving the barrier function of skin or improving the moisturizing capacity of skin may be an embodiment of a method for improving the lamellar structure of skin. A method for improving the moisturizing capacity of skin may be an embodiment of a method for improving the barrier function of skin.

By the method, specifically, by applying the active ingredient to an organism, an effect of improvement in condition of the application target of the active ingredient may be obtained in the organism. Hence, the method may be a method for obtaining an effect of improvement in condition of the application target of the active ingredient.

The effect of improvement in condition of the application target of the active ingredient is as described above. That is, the method may be a method for preventing and/or treating a symptom relating to deterioration of the condition of the application target of the active ingredient. The method may be a method for preventing and/or treating a symptom relating to deterioration of the condition of a body surface, such as skin and body hair. The method may be, particularly, a method for preventing and/or treating a symptom relating to deterioration in skin condition.

The application scheme, for example, application target, application portion, application timing, application period, number of times of application, application amount, and other conditions relating to application, of the active ingredient is not particularly limited, so long as an intended effect is obtained. The application scheme of the active ingredient can be appropriately set according to various conditions such as the type of the active ingredient, the type, age, and health condition of the application target, and the use purpose of the active ingredient.

The active ingredient, for example, may be applied to an organism as it is or may be applied to an organism after being prepared as a composition, such as a cosmetic composition and a pharmaceutical composition, containing the active ingredient. For the composition containing the active ingredient, the descriptions concerning the composition can be similarly applied. In addition, the active ingredient may be applied solely or in combination with another ingredient. For the other ingredient, the descriptions concerning the ingredient other than the active ingredient in the composition can be similarly applied. In addition, examples of the other ingredient also include compositions such as cosmetic compositions and pharmaceutical compositions.

The application target and application portion of the active ingredient are as described above. That is, the active ingredient can be applied to, for example, such an application target as exemplified above. The active ingredient can be applied to, for example, such an application portion as exemplified above. The active ingredient can be applied to the organism by an appropriate means such as, for example, spreading, pasting, and spraying.

The application period of the active ingredient may be, for example, 1 hour or longer, 6 hours or longer, 1 day or longer, 3 days or longer, 1 week or longer, 4 weeks or longer, 2 months or longer, 6 months or longer, 12 months or longer, 2 years or longer, 5 years or longer, or 10 years or longer. The active ingredient may be applied, for example, routinely or only during a specific period. The active ingredient may be applied, for example, continuously or intermittently. The active ingredient may be applied, for example, until an intended effect is obtained. The active ingredient may be applied, for example, once a day, or two or more times a day as divided portions. The active ingredient may be applied, for example, every day, or once in several days. The application amount of the active ingredient at the time of each application may or may not be constant. The application amount of the active ingredient for each application, for example, may be 0.1 μg/cm² or more, 0.5 μg/cm² or more, 1 μg/cm² or more, 5 μg/cm² or more, or 10 μg/cm² or more, may be 500 mg/cm² or less, 100 mg/cm² or less, 50 mg/cm² or less, 10 mg/cm² or less, 5 mg/cm² or less, 1 mg/cm² or less, 0.5 mg/cm² or less, or 0.1 mg/cm² or less, or may be within a range defined as a combination thereof. The application amount of the active ingredient for each application may specifically be, for example, 0.1 μg/cm² to 500 mg/cm².

The active ingredient can be applied to an organism, for example, by using the composition, that is, by applying the composition. That is, an embodiment of the method may be a method including a step of applying the composition to an organism. That is, the term “application of the active ingredient” also includes application of the composition. The application scheme, for example, application target, application portion, application timing, application period, application number of times, application amount, and other conditions relating to application, of the composition is not particularly limited, so long as an intended effect is obtained. The application scheme of the composition can be appropriately set according to various conditions such as the type and amount of the active ingredient, the type(s) and contained amount(s) of other ingredient(s), the type and form (dosage form) of the composition, the type, age, and health condition of the application target, and the purpose of the composition. The aforementioned descriptions concerning the application scheme of the active ingredient can be similarly applied to cases of applying the composition to an organism. That is, the composition can be applied to, for example, such an application target as exemplified above. The composition can be applied to, for example, such an application portion as exemplified above. Furthermore, the application amount of the composition can be set to be an amount such that an application amount of the active ingredient as exemplified above is obtained. In addition, the composition may be used solely or in combination with another ingredient. For the other ingredient, the descriptions concerning the ingredient other than the active ingredient in the composition can be similarly applied. In addition, examples of the other ingredient also include compositions such as cosmetic compositions and pharmaceutical compositions.

### <3>Use of the Active Ingredient

Disclosed herein is the use of the active ingredient for the purpose(s) as exemplified above. That is, for example, use of the active ingredient for improving the condition of the application target of the active ingredient, for example, the application target of the composition, and use of the active ingredient in manufacture of a composition, for example, a cosmetic or pharmaceutical composition, for improving the condition of the application target.

Disclosed herein is the active ingredient for use for the purpose(s) as exemplified above. That is, for example, the active ingredient can be used to improve the condition of the application target of the active ingredient, for example, the application target of the composition, and the active ingredient for use in manufacture of a composition, for example, a cosmetic or pharmaceutical composition for improving the condition of the application target.

### EXAMPLES

The present invention will be more specifically explained with reference to the following non-limiting examples.

### Example 1: Evaluation of Lamellar Structure-Forming Ability

### <Preparation of Samples>

Four ceramide NHs (Table 1) shown in the structural formula (I) below wherein n is

12, 14, 16, or 20 (namely, having an acyl chain length of C14, C16, C18, or C22) were purchased from Tokyo Chemical Industry Co., Ltd.

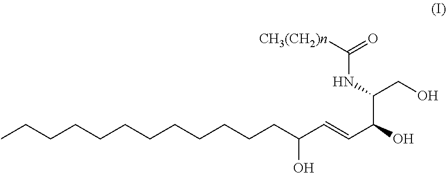

TABLE 1
Ceramide NHs usen in Examples

| | C14- | C16- | C18- | C22- |
|---|---|---|---|---|
| Name | CeramideNH | CeramideNH | CeramideNH | CeramideNH |
| n | 12 | 14 | 16 | 20 |

### <Measurement of Lamellar Structure-Forming Ability>

As an indicator of the lamellar structure-forming ability, Maltese cross patterns were observed via the following procedures. Ceramide NH, cholesterol, and lauric acid were mixed in the mixing ratios shown in Table 2, to prepare samples. Each sample was placed in a glass tube and heated to 90° C. in a thermo shaker (Thermo Shaker for Microtubes, TS-100, Biosan), to melt the entire sample. The melted sample was put to a microscope slide, and a cover slip was placed thereon. The sample was melted again on a hot plate (C-MAGHP4, IKA Japan) at 100° C., set on a polarizing microscope (DX51-P, Olympus), and Maltese cross patterns were observed after the sample had solidified. Observed images were captured with a 20× objective lens and printed on A4 size paper. The number of Maltese cross patterns observed in a 200 μm×200 um area was visually counted.

### <Results>

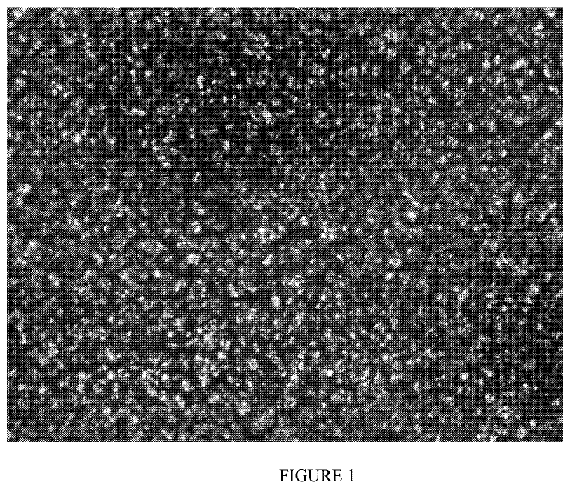

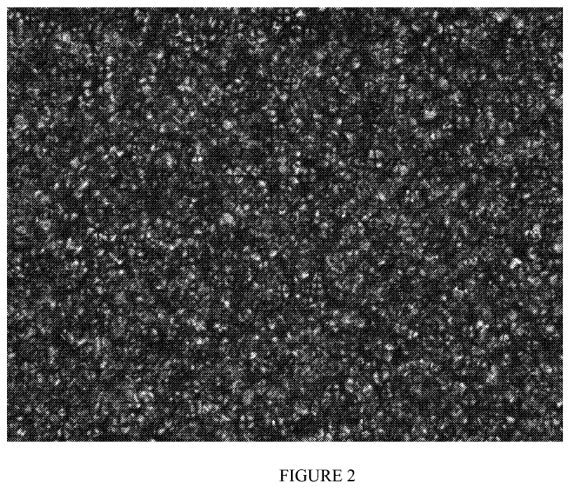

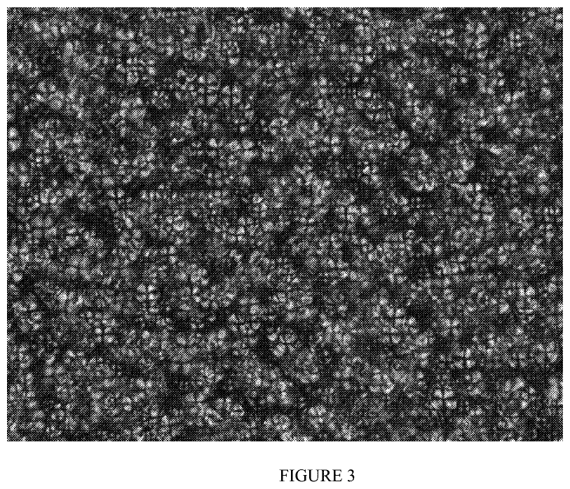

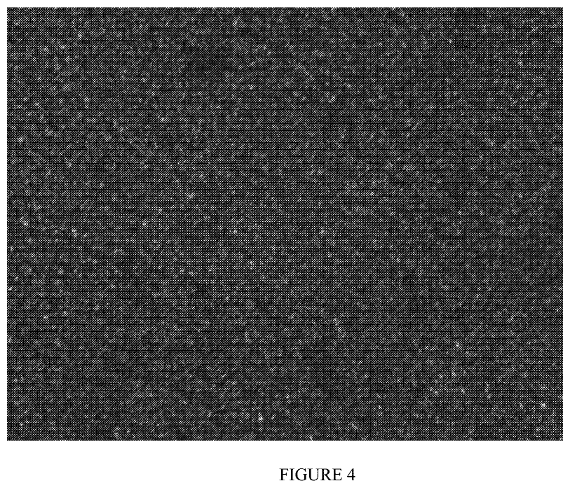

The results of observation of Maltese cross patterns are shown in Table 2 and FIGS. 1 to 4. Ceramide NH having an acyl chain length of C18 (C18-CeramideNH) showed a larger number of Maltese cross patterns (namely, a higher lamellar structure-forming ability) than ceramide NHs having other acyl chain lengths.

TABLE 2
Number of Maltese cross patterns

| | | | | Number of |
|---|---|---|---|---|
| Ceramide NH | | Cholesterol | Lauric acid | Maltese cross |
| | [mg] | [mg] | [mg] | patterns |
| C14-CeramideNH | 10 | 10.09 | 10.1 | 0 |
| C16-CeramideNH | 10.01 | 10.07 | 10.06 | 21 |
| C18-CeramideNH | 10.01 | 10.05 | 10.03 | 137 |
| C22-CeramideNH | 10.01 | 10.06 | 10.07 | 0 |

### Example 2: Evaluation of Effect of Recovering Skin Condition

### <Preparation of Evaluation Samples>

Evaluation samples were obtained by mixing 18 wt % ceramide NH, 7 wt % squalene, 25 wt % triolein, 2 wt % cholesterol sulfate, 14 wt % cholesterol, 5 wt % phosphatidylethanolamine, 4 wt % pristane, and 25 wt % a mixture of fatty acids and fatty acid salts. The mixture of fatty acids and fatty acid salts was prepared by mixing oleic acid (35 wt %), myristic acid (15 wt %), palmitic acid (36 wt %), stearic acid (10 wt %), and linolenic acid (4 wt %) and neutralized with sodium hydroxide by 41%. The mixture of fatty acids and fatty acid salts contains 30 parts by weight of water to 100 parts by weight of fatty acids.

### Measurement of Order Parameter S

The order parameter S was measured, and an effect of recovering skin condition was evaluated, via the following procedures. The order parameter S represents orderliness of array of fatty acid chains, such as orderliness of lamellar structure, and a higher value of the order parameter S indicates that fatty acid chains are arrayed in a more orderly manner. That is, an increase in the order parameter S means improvement in skin condition and may specifically mean improvement in the lamellar structure of skin.
(1) Skin samples (Frozen dermatome human skin, abdominal origin; product number TRA002; BPI) were immersed in a 0.001% 5-DSA (5-doxyl stearic acid) solution for 30 minutes at 37° C., to allow 5-DSA to penetrate the lipid membrane structure. 5-DSA is a labeling agent for ESR measurement.
(2) The skin samples were washed with PBS solution, to remove 5-DSA adhering to the outside of the skin samples.
(3) The skin samples were dried at room temperature and immersed in 5% SLS solution for 1 hour at 37° C., to induce rough skin condition.
(4) After the skin samples were dried at room temperature, the order parameter S was calculated by ESR measurement using a JES-X310 electron spin resonance system (JEOL) and used as “pre-spreading data”. ESR measurement was carried out by a method of a previous report (Mizushima J. et al., Int J Pharm. 2000 Mar. 20; 197(1-2):193-202. Effect of surfactants on human stratum corneum: electron paramagnetic resonance study.), wherein the measurement conditions were Power: 10 mM, Center Field: 336 mT, Sweep: 7.5 mT, Modulation width: 0.5 mT, Scan time: 2 min, Time constant: 0.3 sec (the same shall apply hereafter).
(5) The evaluation sample was spread to the skin sample with a spatula at 3 μg/cm².
(6) The evaluation sample was wiped off from the skin sample, the skin sample was dried at room temperature, and then the order parameter S was calculated by ESR measurement and used as “post-spreading data”.

As a control, the same experiment was conducted without spreading the evaluation

sample.

For each evaluation sample and control, the ratio of post-spreading data to pre-spreading data was calculated and designated as the “recovery rate” of skin condition. The recovery rate for each evaluated sample as a relative value to the recovery rate for the control was calculated and used as the “recovery effect” of the skin condition. The experiment was carried out in duplicate, and the results were obtained as average values.

### <Results>

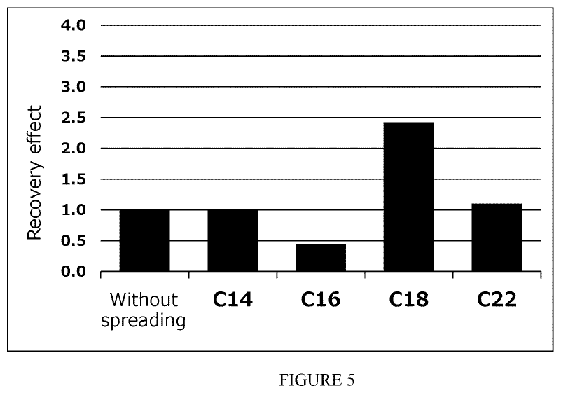

The results are shown in FIG. 5. Ceramide NH having an acyl chain length of C18 (C18-CeramideNH) showed a higher recovery effect of the skin condition than ceramide NHs having other acyl chain lengths.

### INDUSTRIAL APPLICABILITY

According to the present invention, a composition, such as a cosmetic composition, can be provided.

1. A composition for improving condition of an organism's body surface, the composition comprising ceramide NH comprising a C18 acyl chain.

2. The composition according to claim 1, wherein the body surface is skin.

3. The composition according to claim 1, wherein the composition is able to improve a lamellar structure of skin, a barrier function of skin, or a moisturizing capacity of skin.

4. The composition according to claim 1, wherein the composition is able to prevent and/or treat a symptom relating to deterioration in skin condition.

5. The composition according to claim 4, wherein the symptom is selected from the group consisting of rough skin, dry skin, squama (scale), desquamation, dermatitis, psoriasis, and combinations thereof.

6. The composition according to claim 1, wherein the composition is formulated as a cosmetic composition or a pharmaceutical composition.

7. The composition according to claim 1, wherein the composition further contains an ingredient formulated for a cosmetic or pharmaceutical.

8. The composition according to claim 7, wherein the ingredient formulated for a cosmetic or pharmaceutical is selected from the group consisting of a binder, disintegrant, lubricant, stabilizer, diluent, surfactant, pH adjuster, vitamin, mineral, perfume, pigment, preservative, terpenoid, lipid, fatty acid, alcohol, water, antioxidant, anti-inflammatory, moisturizing ingredient, anti-ageing ingredient, anti-cellulite ingredient, skin whitening ingredient, skin tanning ingredient, UV filter, and combinations thereof.

9. The composition according to claim 8, wherein ingredient formulated for a cosmetic or pharmaceutical is selected from the group consisting of a terpenoid, a lipid, a fatty acid, and combinations thereof.

10. The composition according to claim 8, wherein the terpenoid is selected from the group consisting of cholesterol, cholesterol sulfate, squalene, pristane, and combinations thereof.

11. The composition according to claim 8, wherein the lipid is selected from the group consisting of triolein, phosphatidylethanolamine, and combinations thereof.

12. The composition according to claim 8, wherein the fatty acid is selected from the group consisting of lauric acid, myristic acid, palmitic acid, stearic acid, arachidic acid, behenic acid, lignoceric acid, oleic acid, linolenic acid, and combinations thereof.

13. The composition according to claim 1, wherein the ceramide NH comprises a C18:1 alkyl chain and a C18 acyl chain.

14. The composition according to claim 1, wherein the ceramide NH comprises a C18:1 alkyl chain and a C18:0 acyl chain.

15. The composition according to claim 13, wherein the C18:1 alkyl chain has hydroxyl groups at positions C1, C3, and C6 only.

16. A method for improving condition of an organism's body surface, the method comprising applying the composition according to claim 1 to the body surface.
